(19) United States
(12) Patent Application Publication
(10) Pub. No.: US 2007/0078330 A1
(43) Pub. Date: Apr. 5, 2007

(54) Method and apparatus for checking parameters

(76) Inventors: Gabriel Haras, Mucke (DE); Matthias Niethammer, Mohrendorf (DE)

Correspondence Address: HARNESS, DICKEY & PIERCE, P.L.C., P.O.BOX 8910, RESTON, VA, 20195, US

(21) Appl. No.: 11/528,424

(22) Filed: Sep. 28, 2006

(30) Foreign Application Priority Data
Sep. 29, 2005 (DE) 10 2005 046 784.9

(51) Int. Cl. A61B 5/05

(52) U.S. Cl. 600/407

(57) ABSTRACT

A method and an apparatus are disclosed for checking parameters provided for an examination of a patient with the aid of a contrast medium in a contrast medium injector. The parameters are automatically checked for their medical safety for the patient by taking account of at least one individual datum of the patient with the aid of at least one test criterion, and at least one of a warning signal is output, and/or the operation of the contrast medium injector is stopped when a health risk for the patient is determined.

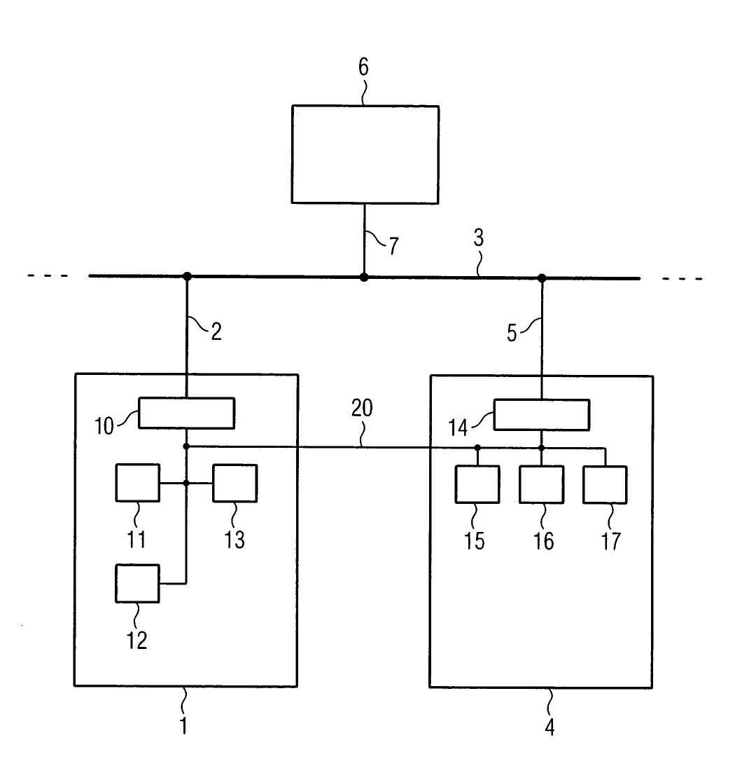

### PRIORITY STATEMENT

The present application hereby claims priority under 35 U.S.C. §119 on German patent application number DE 10 2005 046 784.9 filed Sep. 29, 2005, the entire contents of which is hereby incorporated herein by reference.

### FIELD

The invention generally relates to a method and/or an apparatus for checking parameters for examining a patient with the aid of a contrast medium.

### BACKGROUND

When obtaining image information, in particular of organs and vessels from the interior of a patient's body with the aid of an imaging medical appliance such as an X-ray machine, X-ray computer tomograph, ultrasound appliance or magnetic resonance appliance, use is frequently made of contrast media in order to obtain high contrast images of the desired tissue region from the interior of the patient's body. A suitable contrast medium is injected into the patient's body before images are taken, and a start is made on taking images of the tissue of interest whenever sufficient contrast medium has built up in the tissue of interest such that high contrast images of the tissue of interest can be obtained.

The application of contrast media in medical imaging is, however, not entirely unproblematical, since complications such as allergic reactions, for example, can occur in the event of incompatibility of the contrast medium with a patient. According to an Australian study from 1991 (Intravenous contrast media: use and associated mortality, Med J Aust. 1991 November, 4; 155(9) :618-23), on a statistical basis there are even 13 cases of death per 1 million injections for examinations with the aid of contrast media.

The injection of a contrast medium into the body of a patient is undertaken with the aid of a contrast medium injector that executes an injection protocol input by a user and having injection and/or operating parameters. There is no further monitoring as to extent to which the prescribed parameters of the injection protocol are compatible in health terms for the respective patient.

### SUMMARY

In at least one embodiment of the invention, a method and/or an apparatus is specified, with the aid of which it is possible to lower the complication rate when examining a patient with the aid of a contrast medium.

According to at least one embodiment of the invention, a method is provided for checking parameters provided for an examination of a patient with the aid of a contrast medium in a contrast medium injector, in the case of which the parameters are automatically checked for their medical safety for the patient by taking account of at least one individual datum of the patient with the aid of at least one test criterion, a warning signal being output, and/or the operation of the contrast medium injector being stopped when a health risk for the patient is determined. It is thus proposed that before examining a patient with the aid of a contrast medium the parameters of an injection protocol be automated, preferably with the aid of a number of test criteria, for their compatibility with the patient to be examined, in order, if appropriate, to be able to identify and change the prescribed parameters that are dangerous for the patient's health. The checking of the parameters is performed by taking account, preferably, of a number of individual data of the patient, which are understood to include, for example, the age, the weight, the sex, the body size, the bodily constitution, the disease history, prior examinations etc. of the patient such as are to hand, for example, in a patient file, preferably an electronic patient file. A person undertaking the examination is referred to the problematical parameters with the aid of a warning signal upon establishing a health risk to the patient.

The operation of the contrast medium injector is even preferably stopped until the problematic parameter is selected to provide medical safety for the patient. Thus, the method is a type of control method for examinations with the aid of contrast media, since human error cannot be excluded in selecting suitable parameters for examining a patient. The method according to at least one embodiment of the invention therefore increases patient safety. Since the test criteria are excluded automatically, there is no increase in the outlay on medical staff in the case of such examinations.

In accordance with one variant of at least one embodiment of the invention, the injection or operating parameters for the contrast medium injector comprise the type of the contrast medium, the volume of contrast medium to be injected, the maximum flow rate of the contrast medium, and/or the injection period of the contrast medium. Here, the flow rate is understood as a volume of contrast medium that is injected into a patient per unit of time.

In accordance with one embodiment of the invention, the test criteria to be applied can be fixed by a user. Fixing the test criteria to be applied is preferably done in the form of a table that can be executed during checking of the admissibility of the parameters by a program controlled computation device. It is possible in this case for patient-specific tables to be drawn up individually with the aid of test criteria, or else to establish a general table with test criteria and use these for a multiplicity of patients. The test criteria can in this case include simple rules or else complex associations.

Variants of embodiments of the invention provide that the test criteria generally comprise the age, the body size and/or rules that take account of the weight of the patient. Thus, rules and test criteria can be set up in accordance with which it is permissible to inject per examination only a specific maximum volume of contrast medium into a patient as a function of his age, his body size, his weight and/or his body mass index. If the examination of the patient is carried out with the aid of an X-ray computer tomograph, it is possible in accordance with one variant of the invention to determine the body size and/or the weight of the patient from a topogram of the patient obtained with the aid of an X-ray computer tomograph. Whereas the body size can be taken virtually directly from the topogram, it is possible to infer the weight or the mass of the patient after evaluating the imaged contour of the patient and after evaluating density values derived from the attenuation values.

It can, however, be established that it is impermissible for the flow rate of the contrast medium to exceed a specific maximum value as a function of the age of the patient. If a parameter selected for examining a specific patient breaks one of the rules by lying outside the permissible range when taking account of the individual data of the patient, the person undertaking the examination is advised of the problematic parameter by a warning signal, and/or the operation of the contrast medium injector is stopped until the parameter lies in the range permissible for the patient.

According to further variants of embodiments of the invention, the test criteria include prior medical knowledge of the patient. The prior medical knowledge of the patient can in this case include information of previous examinations with the aid of contrast media, contrast medium allergies knowledge of and/or of physical problems in connection with contrast media, for example renal insufficiency.

Furthermore, according to one variant of an embodiment of the invention, the test criteria can also include general prior medical knowledge of contrast media which is to be understood, for example, as information of fundamental type relating to the contrast medium used and that are independent of the respective patient. Thus, it can also be of interest for the use of a contrast medium whether complications, for example allergies or collapsing patients, have been observed more frequently for a specific type of contrast medium or for the intended concentration to be used for the contrast medium.

To the extent that variants of the apparatus relate to the parameters and the test criteria, the statements made previously in connection with the method according to at least one embodiment of the invention are also valid for the apparatus according to at least one embodiment of the invention.

One embodiment of the apparatus provides that the computation device is part of the contrast medium injector. It is then possible for the checking of the parameters for their health safety for the respective patient to be performed directly in the contrast medium injector. In this case, it would be necessary to be able to input the individual data relating to the patient into the contrast medium injector, or the contrast medium injector would need to have access to the individual data of the patient, for example to a patient file stored in an HIS or RIS system (Hospital Information System and Radiology Information System respectively).

Example embodiments of the invention provide that the computation device is part of an imaging medical appliance connected to the contrast medium injector that is connected as a rule via a network connection to an HIS or RIS system such that it is possible to access the individual patient data present in the patient files. It is therefore possible to check parameters of the contrast medium injector for their health safety for the patient with the aid of the computation device of the medical appliance which, for this purpose, reads in the parameters from the contrast medium injector and uses the test criteria to check the parameters of the contrast medium injector on the basis of the individual patient data required therefore.

According to variants of embodiments of the invention, the medical appliance is an X-ray machine, a C-arc X-ray machine, an X-ray computer tomograph, an ultrasound appliance or a magnetic resonance appliance.

### BRIEF DESCRIPTION OF THE DRAWINGS

An example embodiment of the invention is illustrated in the attached schematic that shows a medical apparatus having an imaging, medical appliance and a contrast medium injector.

### DETAILED DESCRIPTION OF THE EXAMPLE EMBODIMENTS

The terminology used herein is for the purpose of describing particular embodiments only and is not intended to be limiting of the present invention. As used herein, the singular forms “a”, “an” and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described. In the case of the present example embodiment, the medical appliance is an X-ray computer tomograph 1 that is connected in the case of the present example embodiment to a network 3 via a line 2. The network 3 is a network present in a medical institution, for example a hospital or a clinic. The contrast medium injector 4 is likewise connected to the network 3 via a line 5. The situation is the same for an HIS/RIS system 6 that is connected to the network 3 via a line 7.

The X-ray computer tomograph 1 has a computation unit 10, a memory 11, a display apparatus 12 and input device 13, for example in the form of a keypad or a computer mouse. The computation unit 10, the memory 11, the display apparatus 12 and the input device 13 are illustrated only schematically in the figure. The contrast medium injector 4 also includes in the figure, illustrated in a merely schematic fashion, a computation device 14, a memory 15, a display apparatus 16 and input device 17 which are, in turn, input device(s) known per se, for example a keypad, a touch screen, etc.

The aim is to use the X-ray computer tomograph 1 to examine a patient radiologically (not illustrated in the figure) after the injection of a contrast medium with the aid of a contrast medium injection 4. In order to control the injection of the contrast medium into the patient, there is present in the memory 15 of the contrast medium injector 4 an injection program with an injection protocol that has operating parameters for the injection of the contrast medium. The parameters are, inter alia, the type of contrast medium, the volume of the contrast medium to be injected, the maximum flow rate of the contrast medium and the injection period of the contrast medium. The parameters for the injection protocol are input as a rule in advance by a person carrying out the examination, via the input device 17.

Since it is also possible during the radiological examination of a patient under the injection of a contrast medium for complications such as, for example, allergic reactions of the patient to the contrast medium, to occur, it is provided to examine the parameters selected for the examination of the patient for their medical safety for the patient before the injection of the contrast medium into the patient.

In the case of the present example embodiment, there are therefore present in the memory 11 of the X-ray computer tomograph 1 test criteria that include the general rules that take account of the age, the body size or the weight of the patient, and prior medical knowledge of the patient, as well as general prior medical knowledge of contrast media. The person undertaking the examination of the patient can use the input device 13 of the X-ray computer tomograph 1 to fix the test criteria that he would like to use to check the parameters for the examination of the patient. The person undertaking the examination preferably selects from a table relating to available criteria the criteria that he wishes to have checked for a specific patient. The selection of the desired test criteria can be performed in each case before examination of the patient. However, it is also possible, particularly in the case of patients for whom repeated examinations are expected, to draw up a table with test criteria that is adapted to the patient, and to store each of these in a patient's file for the patient, for example in an HIS/RIS system such that they can be called up.

The test criteria can in this case include general rules that take account of the age, the body size or the weight of the patient. It is possible, for example, to fix the following test criteria:
1. Patients under 12 years may be injected at most with 90 ml of a, or a specific, contrast medium.
2. Patients over 12 years may be injected with at most 150 ml of a, or a specific, contrast medium.
3. The maximum flow rate may not exceed 5 ml per second for patients over 60 years.
4. At most f₁(x) ml of a, or a specific, contrast medium may be injected into patients with a mass of x kilograms.
5. At most f₂(Y) ml of a, or a specific, contrast medium may be injected into a patient with a body mass index (BMI) of y.

The data on age and amount, which are based as a rule on empirical values, and the indicated functional relationships are to be understood here only by way of example and not conclusively. The aim is merely to show how rules can be set up by taking account of individual data in order in advance of the examining of a patient with the aid of a contrast medium to be able, by using the individual data of the patient to be examined, to employ the test criteria set up in order to check the parameters selected for the examination, so as to exclude a health risk to the patient.

Furthermore, it is possible to make use of test criteria that take account of prior medical knowledge of the patient to be examined. Such prior medical knowledge is present, for example, in electronic patient files that are stored in HIS/RIS systems. Such prior knowledge includes information relating to earlier examinations with the aid of contrast media which are required, in particular, whenever a patient has already been very frequently examined with the aid of contrast media. It is particularly to be recommended in this case to monitor the overall amount of the contrast medium administered to a patient within a specific time period, for example a day.

One aim thereby is to ensure that the patient does not encounter any physical problems, for example kidney problems, caused by contrast medium. An examination with the contrast medium can therefore not take place again until the contrast medium administered in the course of an earlier examination has been washed out of the body or broken down in the body. Importance attaches, furthermore, in this connection to knowledge of contrast medium allergies of the patient, and of physical problems of the patient that are associated with contrast media such as, for example, renal insufficiency. Further test criteria include general prior medical knowledge of contrast media that are independent of the respective patient, it being possible to check whether complications, for example allergic reactions of patients, have occurred frequently in the case of a specific type or specific concentrations used for a type of contrast medium. Consequently, appropriate test criteria can be used, to monitor the quantity of contrast media administered so far to a patient, or it can be checked that the patient has no allergies to a contrast medium selected for the examination.

If the test criteria have been fixed for a patient and if an examination with the aid of a contrast medium is envisaged for the patient, the computation unit 10 of the X-ray computer tomograph 1 uses a network 3 to request from the contrast medium injector 4 the parameters of the injection protocol that are provided for examining the patient with the aid of the contrast medium. Likewise, if the individual, personal data of the patient are not yet present in the computation unit 10 of the X-ray computer tomograph 1, the network 3 is used to request the individual data of the patient from the HIS/RIS system. It is possible in this context to determine separate individual data of the patient, for example the body size and/or the weight of the patient, from a topogram of the patient produced with the aid of the X-ray computer tomograph.

The body size of the patient may virtually be gathered directly from the topogram. The weight or the mass of the patient can be inferred by using the imaged contour of the patient and by using density values derived from the attenuation values. After the respective data have been supplied or determined, the parameters of the contrast medium injector 4 that are provided for the examination are present, together with the individual data, including prior medical knowledge, of the patient in the X-ray computer tomograph 1 such that the computation device 10 can check the preselected parameters for their medical safety under programmed control with the aid of the test criteria previously set up. An appropriate test program is, moreover, present in the memory 11.

If proceeding from the previously mentioned rules, the computation device tests as a function of the patient's age whether the volume of the contrast medium (parameter) that is to be injected into the patient complies with the rules set up (test criteria). The situation is the same with the rules that take account of the body size, the weight or the sex of the patient and which relate not only to the volume of the contrast medium, but also to the flow rate or other parameters. Furthermore, a check is made with the aid of the individual patient data as to whether the patient has in the past shown allergic reactions to the contrast medium (parameter) selected, or had physical problems.

If the selected test criteria are met, the radiological examination of the patient can be carried out by recourse to the contrast medium with the aid of the X-ray computer tomograph 1 and the contrast medium injector 4, and this is communicated to the person undertaking the examination, for example via the display apparatus 12.

If, however, the existence of health risks for the patient has resulted from checking the parameters, be it that a wrong contrast medium was selected to which the patient reacts allergically, or be it that maximum bounds of volumes referred to the age, the weight or the body size of the patient, were exceeded, or that a health risk for the patient has been determined on the basis of one or more other test criteria, the computation device 10 generates as a rule a warning signal that can, for example, be displayed as an optical warning signal on the display apparatus 12 of the X-ray computer tomograph 1, and additionally on the display apparatus 16 of the contrast medium injector 4.

However, the warning signal need not necessarily be an optical warning signal. Rather, acoustic warning signals or other warning signals also come into consideration. In addition, or as an alternative, the computation device 10 can generate a control signal that is transmitted to the contrast medium injector 4 and stops the contrast medium injector 4 from operating until all the test criteria are fulfilled and medical safety of the parameters has been achieved. In addition to the warning signal, the problematical parameter that violates one of the test criteria is displayed in this case on the display apparatus 12.

The checking of the test criteria that has been outlined for the X-ray computer tomograph 1 can, furthermore, also be carried out by the computation device 14 of the contrast medium injector 4 in the same way.

It is, furthermore, possible directly to connect the X-ray computer tomograph 1 and the contrast medium injector 4 in an additional or alternative fashion by way of a data line 20 in order to enable the direct bidirectional transmission of data between the X-ray computer tomograph 1 and the contrast medium injector 4.

The invention has been explained above with reference to the example of an X-ray computer tomograph. However, instead of using the X-ray computer tomograph it is also possible to use another imaging medical appliance, for example an X-ray machine, a C-arc X-ray machine, and ultrasound appliance or a magnetic resonance appliance.

The parameters mentioned above for the contrast medium injector, and the test criteria are, however, understood only by way of example and not as final. Accordingly, it is also possible to check yet further parameters and to set up further test criteria.

Not only humans, but also animals come into consideration as patients.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for checking parameters provided for an examination of a patient with the aid of a contrast medium in a contrast medium injector, the method comprising:
automatically checking the parameters for medical safety for the patient by taking account of at least one individual datum of the patient with the aid of at least one test criterion; and
at least one of outputting a warning signal and stopping operation of the contrast medium injector, when a health risk for the patient is determined based upon the automatic checking.

2. The method as claimed in claim 1, wherein the parameters for the contrast medium injector include at least one of a type of the contrast medium, a volume of contrast medium to be injected, a maximum flow rate of the contrast medium, and an injection period of the contrast medium.

3. The method as claimed in claim 1, wherein the test criteria is fixable by a user.

4. The method as claimed in claim 1, wherein the test criteria generally comprise at least one of age, body size and rules that take account of weight of the patient.

5. The method as claimed in claim 4, wherein, in accordance with one or more test criteria, only a specific maximum volume of contrast medium is injectable, per examination, into a patient as a function of at least one of age, body size, weight and body mass index.

6. The method as claimed in claim 4, wherein at least one of the body size and the weight of the patient are determined from a topogram of the patient obtained with the aid of an X-ray computer tomograph.

7. The method as claimed in claim 4, wherein, in accordance with a test criterion, the flow rate of the contrast medium can exceed a specific maximum value as a function of the age of the patient.

8. The method as claimed in claim 1, wherein the test criteria include prior medical knowledge of the patient.

9. The method as claimed in claim 8, wherein the prior medical knowledge of the patient includes information relating to previous examinations with at least one of the aid of contrast media, knowledge of contrast medium allergies and physical problems.

10. The method as claimed in claim 1, wherein the test criteria include general prior medical knowledge of contrast media.

11. An apparatus for checking parameters provided for examination of a patient with the aid of a contrast medium in a contrast medium injector, the apparatus comprising:
a contrast medium injector; and
a computation device to automatically check the parameters for medical safety for the patient by taking account of at least one individual datum of the patient with the aid of at least one test criterion, the computation device further being useable to generate at least one of an outputtable warning signal and a control signal that stops the operation of the contrast medium injector, when a health risk for the patient is determined based upon the automatic checking.

12. The apparatus as claimed in claim 11, wherein the parameters for the contrast medium injector include at least one of a type of contrast medium, a volume of contrast medium to be injected, a maximum flow rate of the contrast medium, and an injection period of the contrast medium.

13. The apparatus as claimed in claim 11, wherein the test criteria is fixable by a user with the aid of at least one input device connected to the computation device.

14. The apparatus as claimed in claim 11, wherein the test criteria generally comprise at least one of age, body size and rules that take account of the weight of the patient.

15. The apparatus as claimed in claim 14, wherein, in accordance with one or more test criteria, only a specific maximum volume of contrast medium, per examination, is injectable into a patient as a function of at least one of age, body size, weight and body mass index.

16. The apparatus as claimed in claim 14, wherein, in accordance with a test criterion, flow rate of the contrast medium can exceed a specific maximum value as a function of the age of the patient.

17. The apparatus as claimed in claim 11, wherein the test criteria include prior medical knowledge of the patient that is stored in a patient file.

18. The apparatus as claimed in claim 17, wherein the prior medical knowledge of the patient includes at least one of information relating to previous examinations with the aid of contrast media, knowledge of contrast medium allergies and physical problems.

19. The apparatus as claimed in claim 11, wherein the test criteria include general prior medical knowledge of contrast media.

20. The apparatus as claimed in claim 11, wherein the computation device is part of the contrast medium injector.

21. The apparatus as claimed in claim 11, wherein the computation device is part of an imaging medical appliance connected to the contrast medium injector.

22. The apparatus as claimed in claim 21, wherein the imaging medical appliance is at least one of an X-ray machine, an X-ray computer tomograph, a C-arc X-ray machine, an ultrasound appliance and a magnetic resonance appliance.

23. The apparatus as claimed in claim 22, wherein at least one of body size and weight of the patient is determinable from a tomogram of the patient obtained with the aid of an X-ray computer tomograph.

24. The method as claimed in claim 2, wherein the test criteria is fixable by a user.

25. The method as claimed in claim 2, wherein the test criteria generally comprise at least one of age, body size and rules that take account of weight of the patient.

26. The method as claimed in claim 25, wherein, in accordance with one or more test criteria, only a specific maximum volume of contrast medium is injectable, per examination, into a patient as a function of at least one of age, body size, weight and body mass index.

27. The apparatus as claimed in claim 12, wherein the test criteria is fixable by a user with the aid of at least one input device connected to the computation device.

28. An apparatus for checking parameters provided for an examination of a patient with the aid of a contrast medium in a contrast medium injector, the apparatus comprising:
means for automatically checking the parameters for medical safety for the patient by taking account of at least one individual datum of the patient with the aid of at least one test criterion; and
means for at least one of outputting a warning signal and stopping operation of the contrast medium injector, when a health risk for the patient is determined based upon the automatic checking.

29. The apparatus as claimed in claim 28, wherein the imaging medical appliance is at least one of an X-ray machine, an X-ray computer tomograph, a C-arc X-ray machine, an ultrasound appliance and a magnetic resonance appliance.

30. The apparatus as claimed in claim 29, wherein at least one of body size and weight of the patient is determinable from a tomogram of the patient obtained with the aid of an X-ray computer tomograph.

31. A computer readable medium including program segments for, when executed on a computer device, causing the computer device to implement the method of claim 1.
